(19) United States
(12) Patent Application Publication
(10) Pub. No.: US 2024/0285163 A1
(43) Pub. Date: Aug. 29, 2024

(54) SYSTEM FOR MEASUREMENT OF PERIPHERAL ABERRATION

(71) Applicant: UNIVERSITY COLLEGE DUBLIN, Dublin (IE)

(72) Inventors: Fengzhou Fang, Dublin (IE); Yanbo Zhao, Dublin (IE)

(73) Assignee: UNIVERSITY COLLEGE DUBLIN, Dublin (IE)

(21) Appl. No.: 18/570,445

(22) PCT Filed: Jun. 29, 2022
(86) PCT No.: PCT/EP2022/067993

§ 371 Date: Dec. 14, 2023

(30) Foreign Application Priority Data
Jun. 30, 2021 (GB) 2109423.0

(51) Int. Cl. A61B 3/10; A61B 3/00

(52) U.S. Cl. CPC A61B 3/1015; A61B 3/0058

(57) ABSTRACT

The present invention relates to a system for measurement of peripheral aberration of the human eye. The system is characterized by a specially designed multifaced mirror having its each face distributed along an elliptical contour. The normal to any point on the elliptical contour bisects an angle formed by the point and the line connecting the two focal points of the elliptical contour. The length of the multifaced mirror is equal to the length of a line segment formed between the intersections of a plurality of tangents drawn at each intersection of the elliptical contour and a plurality of rays. The plurality of rays extends from a focal point at the centre of the entrance pupil to a plurality of other focal points at the centre of the entrance pupil. Each ray has a predetermined difference in gradient with respect to each other adjacent ray.

### FIELD

The present disclosure relates to a system and method for measurement of peripheral aberration.

### BACKGROUND

Until the recent past, conventional aberrometers were configured to merely measure on-axis aberration. Of late, it was observed by researchers that peripheral focus state plays an important role in development of myopia, and the first contact lens that focused peripheral images in front of the retina to slow progression of myopia was introduced in the market.

The known means for measurement of peripheral aberration includes use of autorefractometers and use of wavefront sensor based aberrometers. Autorefractometers known in the art take longer time for measurement of peripheral aberration since its measuring optical path is fixed, and it requires an individual to change the orientation of his/her head or eye for each measurement. Further, in comparison to conventional aberrometers, the scale of information captured by autorefractometers is much lesser, and only lower order aberrations such as defocus and astigmatism or in other words only first and second order of Zernike polynomials are captured.

Conventional aberrometers are mostly based on Hartmann-Shack wavefront sensors, which convert light carrying wavefront information coming out of the eye, into a set of spots or data points on the digital camera. The spots are created when micro lenses focus the light onto a camera placed in the focal plane of the micro lens. Conventional aberrometers are however not capable of shining laser beam into the human eye from different directions and receive the reflected light and further not capable of achieving multi-meridional peripheral aberration measurement.

There is therefore an unfulfilled and unresolved need in the art for a system for efficient measurement of peripheral aberration of the human eye, and this forms the primary objective of the present invention.

### SUMMARY OF INVENTION

The present invention relates to a system comprising a multifaced mirror specially constructed for efficient measurement of peripheral aberration of the human eye, as set out in the appended claims.

In a preferred embodiment of the present invention, a system for measurement of peripheral aberration is provided. The system comprises a multifaced mirror, at least one scanner operably coupled to the multifaced mirror, one or more wavefront sensors mounted on a focal plane of the multifaced mirror, a computing device operably coupled to the wavefront sensor and scanner and configured to receive a plurality of inputs from the wavefront sensor and to alter the orientation of the galvanometer scanner, and an infrared light source operably coupled to the computing device.

The multifaced mirror is specially designed such that each face of the multifaced mirror is distributed along an elliptical contour and along a measured centre of the entrance pupil of the human eye. The length of the multifaced mirror is equal to the length of a line segment formed between the intersections of a plurality of tangents drawn at each intersection of the elliptical contour and a plurality of rays wherein the plurality of rays extends from a focal point at the centre of the entrance pupil to a plurality of other focal points at the centre of the entrance pupil. Each ray has a predetermined difference in gradient with respect to each other adjacent ray. The centre of the entrance pupil and the centre of rotation of the scanner are located at the two focal points of the elliptical contour, and the normal to any point on the elliptical contour bisects an angle formed by the point and the line connecting the two focal points of the elliptical contour.

In an embodiment the scanner comprises a galvanometer scanner. In another embodiment the scanner can be selected from one of: polygon, MEMS scanner and any other laser-beam steering or scanning devices.

In an embodiment of the present invention the predetermined difference in gradient is five degrees.

The computing device further comprises a user interface configured to display a plurality of spot field images, a plurality of real time reconstructed multi-dimensional wavefront images, a plurality of real time images of the pupil of the human eye, Fourier optometric coefficients at different visual angles, and Zernike coefficients at different visual angles. The user interface is also configured to enable multiple modes for measurement of peripheral aberration, such as single point measurement and fast measurement.

The construction and design of the multifaced mirror as per the present invention allows efficient measurement of peripheral aberration. The most important advantage is to ensure efficient measurement while the optical path volume is also small, making it easy to integrate with other functions, like Placido disk and Scheimpflug imaging. The present invention is capable of large scale industrial application, particularly in organizations engaged in research and development of visual optics, spectacle stores, and ophthalmology clinics. The large scale diagnostic data generated by the present invention can be used to train machine learning models to achieve auto-diagnosis of peripheral aberration.

The present invention hence provides a robust and cost-effective solution to drawbacks identified in the art.

In another embodiment there is provided system for measurement of peripheral aberration of a human eye, the system comprising:
a mirror adapted to be moved between different locations;
a scanner operably coupled to the mirror;
one or more wavefront sensors mounted on the conjugate plane of a measured human eye entrance pupil plane;
a computing device operably coupled to the wavefront sensor and the mirror, the computing device configured to receive a plurality of inputs from the wavefront sensor and to alter the orientation of the scanner; wherein.
each position of the mirror is distributed along an elliptical contour with the centre of an entrance pupil of the human eye located at the focal point of the ellipse.

In another embodiment there is provided a method for measuring peripheral aberration of a human eye, the method comprising:
coupling a multifaced mirror with a scanner;
mounting one or more wavefront sensors on the conjugate plane of a measured human eye entrance pupil plane;
configuring a computing device to receive a plurality of inputs from the wavefront sensor and to alter the orientation of the scanner; wherein
each face of the multifaced mirror is distributed along an elliptical contour with the centre of an entrance pupil of the human eye located at the focal point of the ellipse.

There is also provided a computer program comprising program instructions to configure the computing device for causing a computer program to carry out the above method which may be embodied on a record medium, carrier signal or read-only memory.

### BRIEF DESCRIPTION OF DRAWINGS

The invention will be more clearly understood from the following description of an embodiment thereof, given by way of example only, with reference to the accompanying drawings, in which:—

FIG. 1 is a schematic drawing of the multifaced mirror as per a preferred embodiment of the present invention.

FIG. 2 is a schematic drawing of the multifaced mirror arranged along an elliptical contour as per a preferred embodiment of the present invention.

FIG. 3 is a three-dimensional diagram of the multifaced mirror as per a preferred embodiment of the present invention.

FIG. 4 is a real image of a multifaced mirror as per a preferred embodiment of the present invention.

FIG. 5 is a real image of the multifaced mirror and scanner assembled in a system for measurement of peripheral aberration, as per a preferred embodiment of the present invention.

FIG. 6 is a real image of the scanner assembled in a system for measurement of peripheral aberration, as per a preferred embodiment of the present invention.

FIG. 7 illustrates an experimental setup in a laboratory constructed with 30 mm cage system.

FIG. 8 is schematic drawing of a user interface of the computing device, as per a preferred embodiment of the present invention.

### DETAILED DESCRIPTION OF DRAWINGS

The present invention relates to a system for measurement of peripheral aberration, and more particularly to a system comprising a multifaced mirror specially constructed for efficient measurement of peripheral aberration of the human eye.

The system comprises a multifaced mirror, a scanner operably coupled to the multifaced mirror, one or more wavefront sensors mounted on a focal plane of the multifaced mirror, a computing device operably coupled to the wavefront sensor and the multifaced mirror, and one or more infrared light sources and an image capturing means operably coupled to the computing device. Suitably the scanner is a galvanometer scanner. The computing device is configured to receive a plurality of inputs from the wavefront sensor, and to alter the orientation of the galvanometer scanner. The wavefront sensor could be for example, a Hartmann-Shack sensor.

For this application, the system is designed for human eye aberration measurement. The position of the wavefront sensor is relative to the entrance pupil plane (the plane where the wavefront is measured) of human eye.

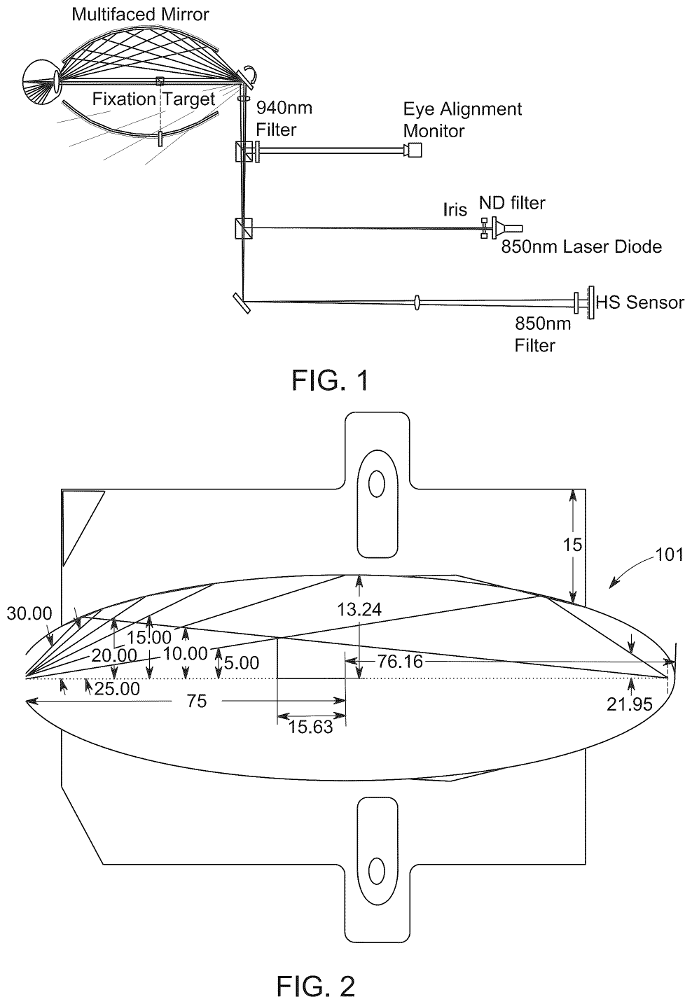

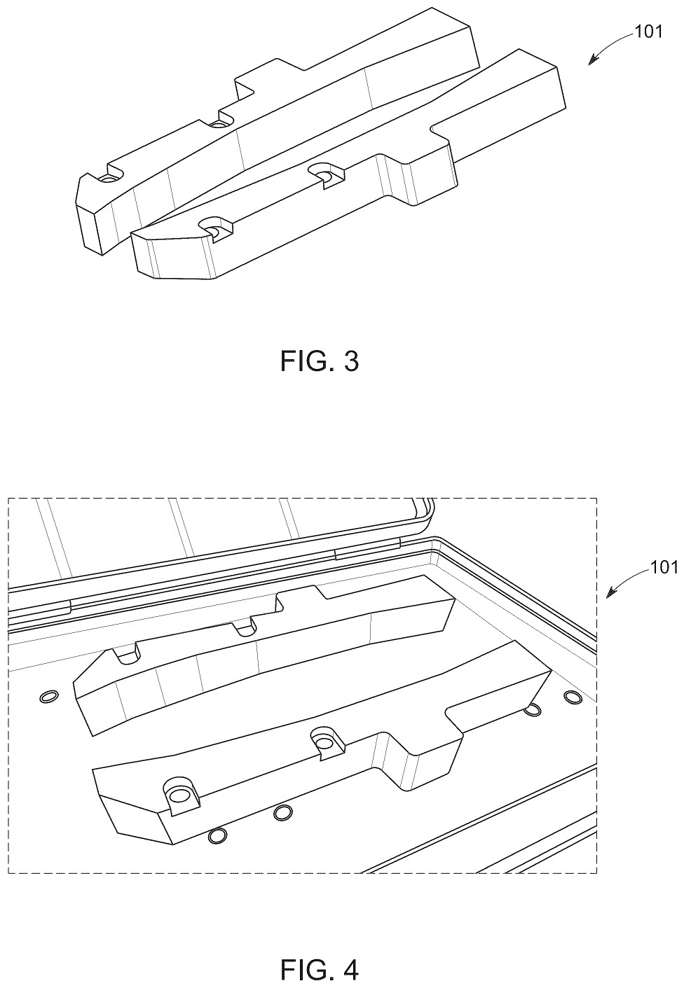

Referring to FIG. 1 and FIG. 2, each face of the multifaced mirror 101 is distributed along an elliptical contour. The centre of the entrance pupil and the centre of rotation of the galvanometer scanner are located at the two focal points of the elliptical contour. The normal to any point on the elliptical contour bisects an angle formed by the point and the line connecting the two focal points of the elliptical contour. This construction ensures a constant optical path. The length of the multifaced mirror 101 is equal to the length of a line segment formed between the intersections of a plurality of tangents drawn at each intersection of the elliptical contour and a plurality of rays.

The plurality of rays extends from a focal point at the centre of the entrance pupil to a plurality of other focal points at the centre of the entrance pupil.

As shown in FIG. 1 and FIG. 2, each ray has a difference in gradient of five (5) degrees with respect to each other adjacent ray. The multifaced mirror 101 is designed based on the optical angle of the scanner, the focal length of the lens closest to the human eye, and the visual field angles to be measured using the system.

It will be appreciated that the shape of the multifaceted mirror can be specifically designed, depending on the application required. Three factors can be taken into consideration in the design, namely the optical angle of the scanner, the focal length of the lens closest to the human eye and the visual field angles to be measured. To guarantee the constant optical path, each face of the mirror is distributed along an elliptical contour and the entrance pupil center, and the center of the entrance pupil of the human eye and the center of rotation of the galvanometer are located at the two focal points of this elliptical contour. The orientation of each mirror is based on the property of the ellipse: the normal to any point on the ellipse is the bisector of the angle formed by the point on the ellipse and the line connecting the two focal points. The length of each mirror is determined by the following method. First, rays are made from the focal point at the entrance pupil centre of the human eye towards another focal point at intervals of 5 degrees. Then, draw a tangent at the intersection of the ray and the ellipse, and the length of the line segment formed by the intersection between the tangents is the length of a single mirror.

This optical path diagram shows the relation of the hardware. Two laser sources are shown in this system, one is 850 nm, the other one is 940 nm. 850 nm infrared light in this picture is for measuring (the thin beam goes into the eye and to be received by wavefront sensor), while laser source 102 is 940 nm, that's for the illumination of human eye front surface, so that the image of the eye from pupil camera to make alignment can be seen.

However, the number of light sources is not limited to two. It is envisaged that Scheimpflug imaging and Placido disk can be added to the system, according to one embodiment of the present invention. Scheimpflug imaging requires 475 nm blue slit LED light, Placido disk needs 625 nm red surface light source (LED array).

FIG. 3 illustrates a three-dimensional view of the multifaced mirror 101 displaying its elliptical contour, and FIG. 4 is a real image of the multifaced mirror 101 as per a preferred embodiment of the present invention.

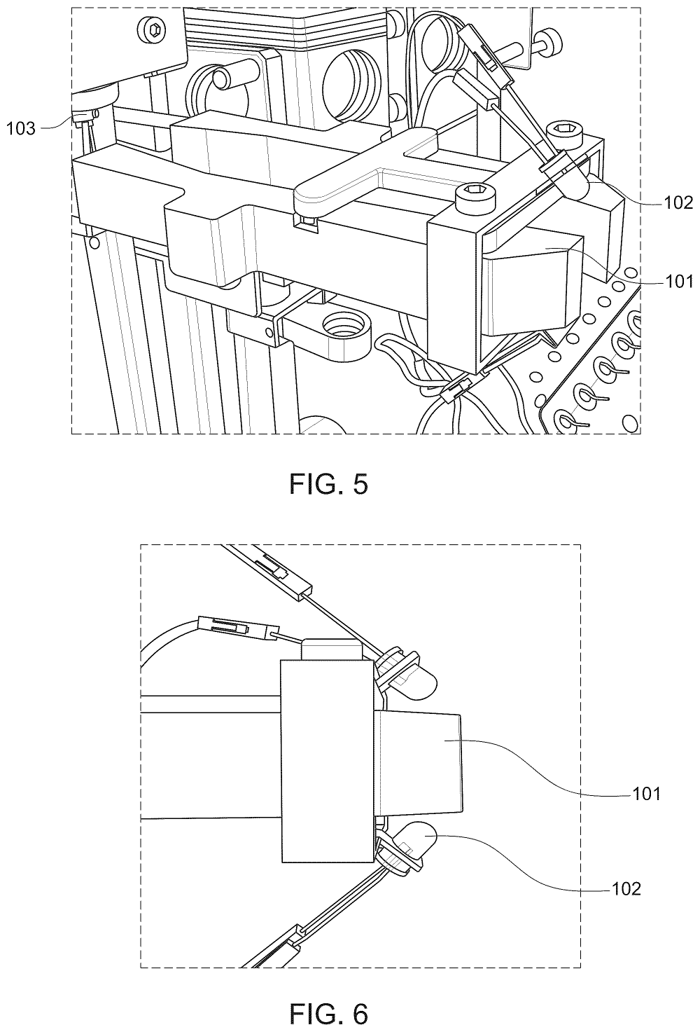

FIG. 5 illustrates the multifaced mirror 101 and galvanometer scanner 103 assembled in a system for measuring peripheral aberration, as per a preferred embodiment of the present invention. FIG. 6 shows a pair of infrared sources 102 arranged above and below the one end of the multifaced mirror 101 for eye front surface illumination. When the system is actuated, a thin beam of infrared light in projected into the fundus of the human eye. Galvanometer scanner 103 directs thin laser beam to certain reflective surface on the mirror then to peripheral retina. A backscattering light from the eye which carries wavefront information, is reflected by the multifaced mirror 101 then galvanometer scanner 103 and is captured by the wavefront sensor. The peripheral aberration is measured from the spot pattern on a detector of the wavefront sensor.

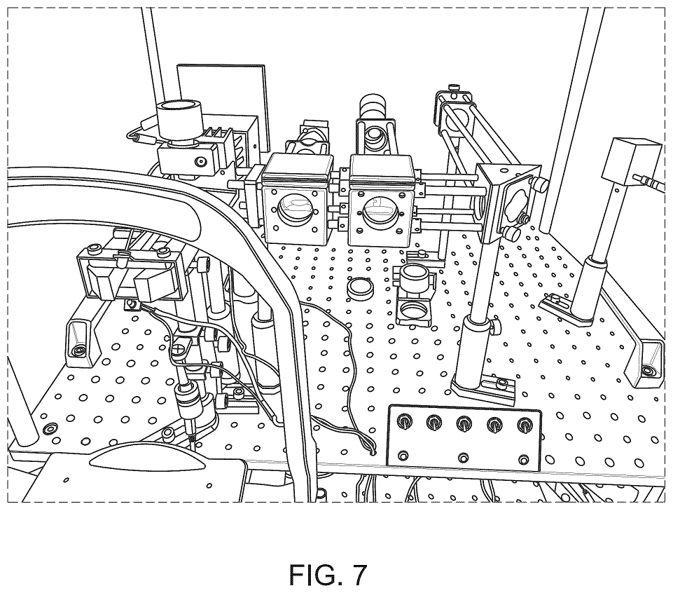

FIG. 7 illustrates an experimental setup in a laboratory constructed with 30 mm cage system. This frame is reliable and popular in lab-based optical path as well as some commercial imaging systems. By building such a set of light paths, the measurement scheme designed in FIG. 1 is realized. A thin laser beam with 0.5 mm diameter shins from the 850 nm continuous compact laser diode. The beam reflected by beam splitter and galvanometer scanner goes into the eye. Due to reflectance of retina, the outcoming wide beam carries the aberrations goes to HS sensor. The wavefront at the entrance pupil plane is conjugated by a pair of relay lenses (200 mm FL and 250 mm FL lenses with 650-1050 nm AR coating) onto the micro lens array of HS sensor. HS sensor captures the spotfield image, all the wavefront information is derived from spot distribution. Sometimes the cornea reflection appears on HS sensor, which is undesired. Polarizing light technique can effectively reduce the reflection from cornea and other optical elements. At the meantime, the front surface of the eye is illuminated by a pair of 940 nm TO1 3/4 housing LEDs to ensure enough energy received by pupil camera for imaging and alignment. A chin & forehead rest mounted on X-Y manual translation stage to fix subject's head.

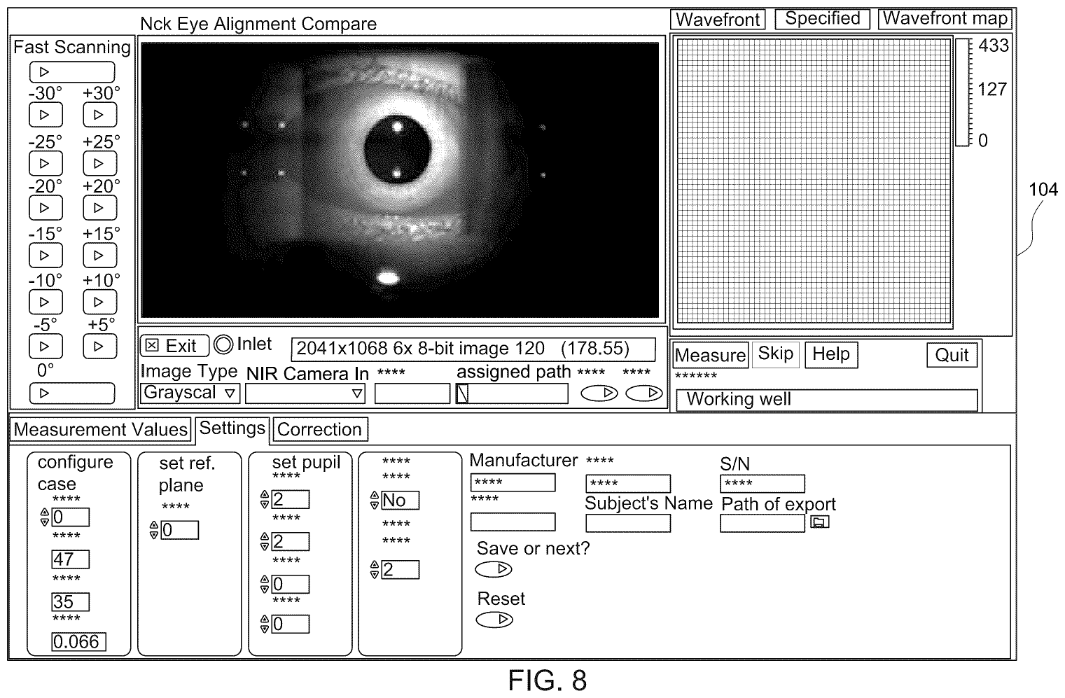

FIG. 8 is schematic drawing of a user interface 104 of the computing device. The user interface 104 is configured to display a plurality of spot field images, a plurality of real time reconstructed multi-dimensional wavefront images, a plurality of real time images of the pupil of the human eye, Fourier optometric coefficients at different visual angles, and Zernike coefficients at different visual angles. The user interface 104 is also configured to enable multiple modes for measurement of peripheral aberration, such as single point measurement and fast measurement. The user interface 104 also provides means for report generation. The generated report comprises details such as a subject's personal information, time stamp, wavefront statistics, Fourier optometric coefficients, and Zernike coefficients at different visual field angles.

The software executable on the system can be based on LabVIEW. The software controls the pupil camera, scanning mirror and wavefront sensor. The user interface allows different modes of measurement: single position measurement and fast measurement. Besides, it should display the spot field image, reconstructed 3D & 2D wavefront surface and pupil image in real time. Also, it is required to show the Fourier optometric and Zernike coefficients.

As for the software structure, the software comprises of three modules: the main functional module, the pupil monitoring module and the wavefront sensor module, which are executed in parallel. In main functional module it communicates with data acquisition card to send a command signal to steer the scanner mirror to a desired angle and read the wavefront sensor. It can also generate a report if the save button is lightened. The main functional module and wavefront sensor module are developed based on state machine structure, which is a very commonly used structure.

As for data export, a sub-VI is developed using report generation module. It shows the subject's personal information, time stamp, wavefront statistics, Fourier optometric and Zernike coefficients at different visual field angles.

It will be appreciated that the multifaced mirror could be alternated by one or more rotatable mirrors operably movable along an elliptical trajectory or galvanometer scanners operably movable along an elliptical trajectory. All the modifications using the property of ellipse or elliptical element to ensure equal optical path of the scanning are within the scope of the present invention. These alternations are not departing from the spirit or scope of the present invention as defined.

It will be further appreciated is designed to measure the peripheral aberrations of the human eye. But its function is not limited to peripheral aberration measurement. The modifications that integrate peripheral aberration measurement with other functions are not departing from the spirit or scope of the present invention as defined.

It will be appreciated that the system is designed to measure human eye. However, system can be used to measure some other optical elements.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present invention as defined.

Further, a person ordinarily skilled in the art will appreciate that the various illustrative method steps described in connection with the embodiments disclosed herein may be implemented using electronic hardware, or a combination of hardware and software. To clearly illustrate this interchangeability of hardware and a combination of hardware and software, various illustrations and steps have been described above, generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention.

In the specification, the terms “comprise, comprises, comprised and comprising” or any variation thereof and the terms “include, includes, included and including” or any variation thereof are considered to be totally interchangeable, and they should all be afforded the widest possible interpretation and vice versa.

The invention is not limited to the embodiments hereinbefore described but may be varied in both construction and detail.

1. A system for measurement of peripheral aberration of a human eye, the system comprising:
a multifaced mirror or a mirror moved between different locations;
a scanner operably coupled to the multifaced mirror;
one or more wavefront sensors mounted on the conjugate plane of a measured human eye entrance pupil plane;
a computing device operably coupled to the wavefront sensor and the multifaced mirror, the computing device configured to receive a plurality of inputs from the wavefront sensor and to alter the orientation of the scanner; wherein.
each face of the multifaced mirror is distributed along an elliptical contour with the centre of an entrance pupil of the human eye located at the focal point of the ellipse.

2. The system as claimed in claim 1, wherein the length of the multifaced mirror is equal to the length of a line segment formed between the intersections of a plurality of tangents drawn at each intersection of the elliptical contour and a plurality of rays, the plurality of rays extending from a focal point at the centre of the entrance pupil to a plurality of other focal points at the centre of the entrance pupil.

3. The system as claimed in claim 1 or 2 wherein each ray has a predetermined difference in gradient with respect to each other adjacent ray, and wherein the centre of the entrance pupil and the centre of rotation of the scanner are located at the two focal points of the elliptical contour, and the normal to any point on the elliptical contour bisects an angle formed by the point and the line connecting the two focal points of the elliptical contour.

4. The system as claimed in any of the preceding claims wherein the predetermined difference in gradient is five degrees or a selected gradient depending on a specific requirement.

5. The system as claimed in any of the preceding claims, wherein the computing device further comprises a user interface configured to display: a plurality of spot field images; a plurality of real time reconstructed multi-dimensional wavefront images; a plurality of real time images of the pupil of the human eye; Fourier optometric coefficients at different visual angles; and Zernike coefficients at different visual angles.

6. The system as claimed in claim 5, wherein the user interface is further configured to enable multiple modes for measurement of peripheral aberration.

7. The system as claimed in any of the preceding claims, further comprising one or more infrared light sources and an image capturing means, operably coupled to the computing device.

8. The system as claimed in any of the preceding claims wherein the multifaced mirror is alternated by one or more rotatable mirrors operably movable along an elliptical trajectory or scanners operably movable along an elliptical trajectory.

9. The system as claimed in any of the preceding claims wherein the multifaced mirror is alternated by one or more scanners operably movable along an elliptical trajectory.

10. A multifaced mirror for measurement of peripheral aberration of a human eye, wherein each face of the multifaced mirror is distributed along an elliptical contour; and
the length of the multifaced mirror is equal to the length of a line segment formed between the intersections of a plurality of tangents drawn at each intersection of the elliptical contour and a plurality of rays, the plurality of rays extending from a focal point at the centre of the entrance pupil towards a plurality of the other focal point,
wherein each ray has a predetermined difference in gradient with respect to each other adjacent ray, and wherein the centre of the entrance pupil is located at one of the two focal points of the elliptical contour, and the normal to any point on the elliptical contour bisects an angle formed by the point and the line connecting the two focal points of the elliptical contour.

11. The multifaced mirror as claimed in claim 10, wherein each ray has a predetermined difference in gradient with respect to each other adjacent ray, and wherein the centre of the entrance pupil is located at the two focal points of the elliptical contour, and the normal to any point on the elliptical contour bisects an angle formed by the point and the line connecting the two focal points of the elliptical contour.

12. The multifaced mirror as claimed in claim 10 or 11 wherein the predetermined difference in gradient is five degrees or a selected gradient depending on a specific requirement.
